(19) United States
(12) Patent Application Publication
(10) Pub. No.: US 2011/0118576 A1
(43) Pub. Date: May 19, 2011

(54) Noninvasive fetal blood oxygen monitoring system and associated method

(76) Inventors: Pirooz Eghtesady, Cincinnati, OH (US); R. Scott Baker, Cincinnati, OH (US)

(21) Appl. No.: 12/675,059

(22) PCT Filed: Aug. 28, 2008
(86) PCT No.: PCT/US08/10207

§ 371 Date: Feb. 24, 2010

Related U.S. Application Data
(60) Provisional application No. 60/967,199, filed on Aug. 31, 2007.

(51) Int. Cl. A61B 5/1455

(52) U.S. Cl. 600/338

(57) ABSTRACT

A method for non-invasively measuring the oxygen saturation of an in utero fetus's blood using near-infrared spectroscopy. Exemplary methods include placement of a sensor on the outside of the uterus approximate the placenta. Other exemplary methods include inserting a probe into the uterus. Sensors may be positioned approximate a particular portion of the fetus, such as the brain or kidney, to measure the oxygen saturation within the particular portion of the fetus.

### CROSS REFERENCE TO RELATED APPLICATION

This application claims the benefit of U.S. Provisional Patent Application No. 60/967,199, filed Aug. 31, 2007, which is incorporated by reference.

### BACKGROUND

The present disclosure relates to blood monitoring systems and, more specifically, to non-invasive fetal blood oxygen monitoring systems. In particular, the present disclosure relates to methods of using a near-infrared spectroscopy (“NIRS”) device operatively coupled to a sensor mounted to a probe that is placed on or within a uterus to determine the saturation of oxygen within fetal blood.

Current fetal monitoring includes fetal heart rate monitoring, ultrasound, stress-test, amniocentesis, chorionic villus sampling (CVS), and fetal blood sampling. These current methods of fetal monitoring are limited because they are indirect, are not highly sensitive (often reflective of terminal stages of fetal distress), can be intermittent with periods of “silent” loss of information, and are invasive (fetal blood sampling).

### INTRODUCTION TO THE INVENTION

Exemplary embodiments include methods for non-invasively measuring the oxygen saturation of an in utero fetus's blood using near-infrared spectroscopy. Exemplary methods include placement of a sensor on the outside of the uterus approximate the placenta. Other exemplary methods include inserting a probe carrying a sensor into the uterus. Sensors may be positioned approximate a particular portion of the fetus, such as the brain or kidney, to measure the oxygen saturation within the particular portion of the fetus. In exemplary embodiments, the use of NIRS oximetry permits non-invasive, continuous measurement of oxygen saturation in the placenta or elsewhere, thereby monitoring oxygen delivery to the fetus. The use of NIRS oximetry to measure the oxygen saturation in the placenta provides an opportunity for fetal intervention and management, enhancing fetal outcomes and survival.

The oxygen monitoring technology can also be applied to enhance outcomes in fetal surgery. Surgical correction of congenital heart defects is associated with neurological and renal complications in ten or more percent of cases. Episodes of low blood flow (ischemia) to organs during cardiopulmonary bypass can be a cause of the complications as well as other poor outcomes. Periods of organ ischemia and oxygen deprivation can also occur during the post-op recovery period. Monitoring and managing these episodes of regional oxygen deprivation is important and can improve outcomes.

In a first aspect, a method of measuring oxygen concentration within fetal blood may include placing a sensor approximate an outside of a wall of a uterus, the sensor being adapted to be operatively coupled to a near-infrared spectroscopy device; and measuring a saturation of oxygen in blood of a fetus present within the uterus using the near-infrared spectroscopy device and the sensor.

In a detailed embodiment of the first aspect, the step of placing the sensor may include placing the sensor approximate the outside of the wall of the uterus generally opposing a placenta present within the uterus and the step of measuring the saturation of oxygen may include measuring a saturation of oxygen in the fetus's blood present within the placenta.

In another detailed embodiment of the first aspect, the sensor may be a miniature sensor. In a further detailed embodiment, the method may include, prior to the step of placing the sensor, creating a minimally invasive incision and inserting the sensor through the minimally invasive incision. In still a further detailed embodiment, the method may include, prior to the step of placing the sensor, visualizing the uterus using a laparoscope.

In another detailed embodiment of the first aspect, the sensor and the near-infrared spectroscopy device may be adapted to be operatively connected via a wireless data link.

In yet another detailed embodiment of the first aspect, the step of measuring the saturation of oxygen may include continuously measuring the saturation of oxygen during at least a portion of a therapeutic procedure. In a further detailed embodiment, the therapeutic procedure may include placing the fetus on cardiopulmonary bypass.

In another detailed embodiment of the first aspect, the step of placing the sensor may include placing the sensor approximate the outside of the wall of the uterus generally near at least one of a brain and a kidney of the fetus and the step of measuring the saturation of oxygen may include measuring a saturation of oxygen in the fetus's blood present within at least one of the brain and the kidney.

In a second aspect, a method of measuring fetal blood oxygen concentration may include inserting a probe into a uterus, the probe including a sensor adapted to be operatively coupled to a near-infrared spectroscopy device; and measuring a saturation of oxygen in blood of a fetus present within the uterus using the near-infrared spectroscopy device and the sensor.

In a detailed embodiment of the second aspect, the step of inserting the probe may include placing the sensor approximate a placenta present within the uterus and the step of measuring the saturation of oxygen may include measuring a saturation of oxygen in the fetus's blood present within the placenta.

In another detailed embodiment of the second aspect, the sensor may be a miniature sensor. In a further detailed embodiment, the method may include, prior to the step of inserting the probe, creating a minimally invasive incision and the step of inserting the probe may include inserting the probe through the minimally invasive incision. In a still further detailed embodiment, the method may include, prior to placing the sensor approximate the placenta, visualizing the uterus using a laparoscope.

In another detailed embodiment of the second aspect, the sensor and the near-infrared spectroscopy device may be adapted to be operatively connected via a wireless data link.

In yet another detailed embodiment of the second aspect, the step of measuring the saturation of oxygen may include continuously measuring the saturation of oxygen during at least a portion of a therapeutic procedure. In a further detailed embodiment, the therapeutic procedure may include placing the fetus on cardiopulmonary bypass.

In another detailed embodiment of the second aspect, the step of inserting the probe may include placing the sensor approximate at least one of a brain and a kidney of the fetus; and wherein the step of measuring the saturation of oxygen includes measuring a saturation of oxygen in the fetus's blood present within at least one of the brain and the kidney.

In a third aspect, a fetal blood oximetry device may include a near-infrared spectroscopy device and a sensor operatively coupled to the near-infrared spectroscopy device, and the sensor may be adapted for use at least one of on or within a uterus.

In a detailed embodiment of the third aspect, the sensor may include a tissue-contact surface at least partially covered with an adhesive and the adhesive may be moisture-resistant. In another detailed embodiment, the sensor may include a connector interposing the sensor and the near-infrared spectroscopy device and the connector may be moisture-resistant. In yet another detailed embodiment, the device may include a probe adapted to be inserted into a uterus and the sensor may be mounted to the probe. In still another detailed embodiment, the sensor and the near-infrared spectroscopy device may be operatively connected via a wireless data link.

### BRIEF DESCRIPTION OF THE DRAWINGS

The detailed description particularly refers to the accompanying Figures in which:

FIG. 1 is a pictorial representation depicting placement of a sensor on a pregnant uterus;

FIG. 2 is a schematic representation depicting the interface of the mother's and the fetus's circulatory systems;

FIG. 3 is a plot of oxygen saturation measured by direct blood gas measurement versus oxygen saturation measured NIRS oximetry;

FIG. 4 is a plot of partial pressure of oxygen (pO₂) measured by direct blood gas measurement versus oxygen saturation measured by NIRS oximetry;

FIG. 5 is a plot of oxygen saturation as a function of time during a first exemplary trial; and

FIG. 6 is a plot of oxygen saturation as a function of time during a second exemplary trial.

### DETAILED DESCRIPTION

Exemplary embodiments described and illustrated herein include methods of measuring fetal blood oxygen saturation, as well as apparatus for measuring fetal blood oxygen saturation. It will be apparent to those of ordinary skill in the art that the embodiments discussed below are exemplary in nature and may be reconfigured without departing from the scope and spirit of the present invention. However, for clarity and precision, the exemplary embodiments discussed herein may include optional steps, methods, and features that one of ordinary skill should recognize as not being a requisite to fall within the scope of the present invention as defined by the claims.

The disclosure includes the use of near-infrared spectroscopy for the measurement of placental oxygen saturation. For example, exemplary embodiments utilize a Somanetics® INVOS® portable oximeter to non-invasively measure placental (fetal) oxygen saturation.

The use of NIRS oximetry allows non-invasive and continuous measurement of oxygen saturation in the placenta, thereby monitoring oxygen delivery to the fetus. Currently, no other non-invasive method allows measurement of placental (fetal) oxygen saturation. The use of NIRS oximetry for measurement of the oxygen saturation in the placenta provides an opportunity for fetal intervention and management, enhancing fetal outcomes and survival.

The oxygen monitoring technology can also be applied to enhance outcomes in fetal surgery. For example, surgical correction of congenital heart defects is associated with neurological and renal complications in ten or more percent of cases. Episodes of low blood flow (ischemia) to organs during cardiopulmonary bypass can be a cause of the complications as well as other poor outcomes. Periods of organ ischemia and oxygen deprivation can also occur during the post-op recovery period. Monitoring and managing these episodes of regional oxygen deprivation is critical and can improve outcomes.

NIRS can be utilized to measure blood oxygen levels, often referred to as oximetry. NIRS technology is non-invasive and painless. Systemic parameters such as blood pressure, heart rate, electroencephalogram (EEG) and blood gases are typically monitored in conjunction with oximetry. Although the systemic parameters cannot give accurate information about individual organ oxygen levels, NIRS can provide individual organ or “regional” oximetry.

NIRS technology functions by emitting and then measuring the reflection of near-infrared light. Near-infrared light is emitted from the “light source” and harmlessly penetrates tissue and bone. Hemoglobin absorbs this light based on how much oxygen is present (bound). Shallow (30 mm) and deep (40 mm) sensors, for example, continuously measure how much light is reflected back. An algorithm is then used to convert the reflection measurements to oxygen saturation in the tissue.

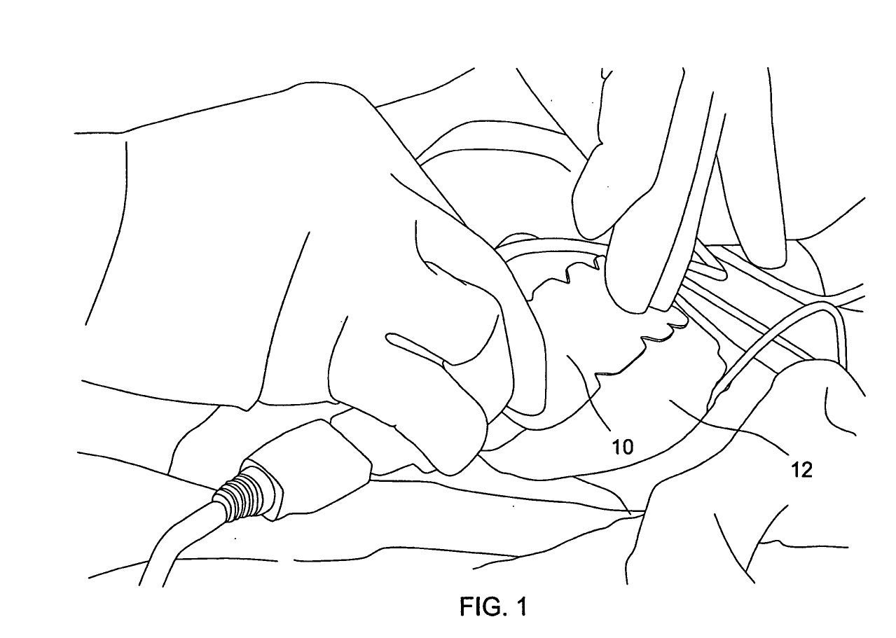

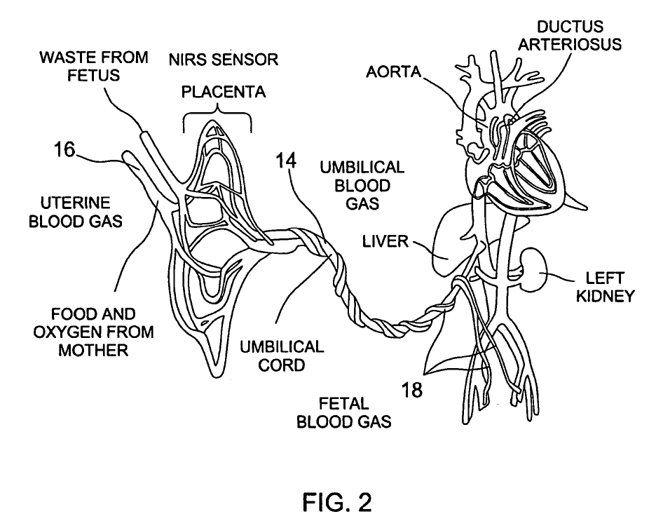

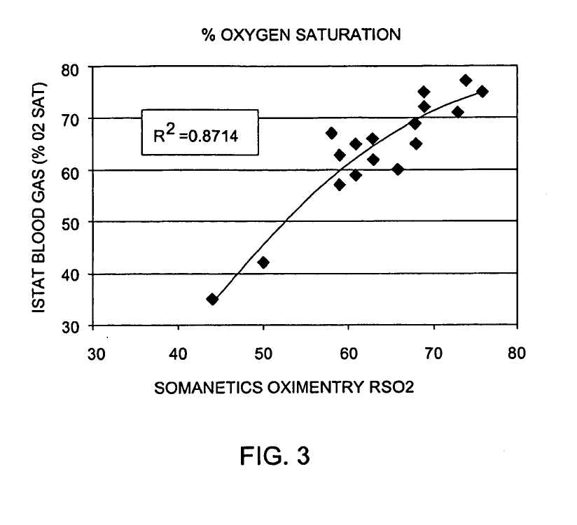

An exemplary embodiment utilizing a Somanetics® INVOS® portable cerebral oximeter is depicted in FIGS. 1 and 2. Data from the exemplary method is shown in FIGS. 3-6. In this exemplary embodiment, the unit is used with disposable adhesive sensors with surface areas less than 20 cm². While some exemplary sensors are sensitive to moisture, it is within the scope of the invention to utilize sensors that are resistant to moisture.

In a study using an exemplary embodiment, four ovine fetuses of 98-110 days gestation were placed on cardiopulmonary bypass for 30 minutes and were followed post-bypass for 2 hours. A NIRS probe (Somanetics® INVOS® 5100B) was placed on the pregnant horn of the ovine uterus to monitor uterine/placental oxygen saturations. The application of the sensor to the uterus is shown in FIG. 1. Used in this manner, the sensors 10 do not injure the uterine surface 12 or interfere with surgical protocol. NIRS values were then compared to oxygen saturations simultaneously obtained by direct blood gas sampling from the umbilical vein 14, uterine vein 16, and fetal arterial circulation 18. These points of direct blood gas sampling are indicated in FIG. 2. Finally, the NIRS values were correlated to the measured blood gases and umbilical blood flows using the best-fit method.

Analysis of the data reveals that the NIRS-derived placental oxygen saturations were positively and tightly correlated with the directly measured umbilical venous oxygen saturations (R2=0.87) and partial pressure of oxygen (pO₂) (R2=0.78), and declining umbilical venous pCO₂ (R2=0.54) and pH (R2=0.65), but not with uterine venous oxygen saturations. NIRS correlated with rising fetal arterial oxygen saturations (R2=0.45) and pO₂ (R2=0.48), and declining pH (R2=0.56) and pCO₂ (R2=0.28). NIRS correlated with umbilical blood flow (R2=0.47).

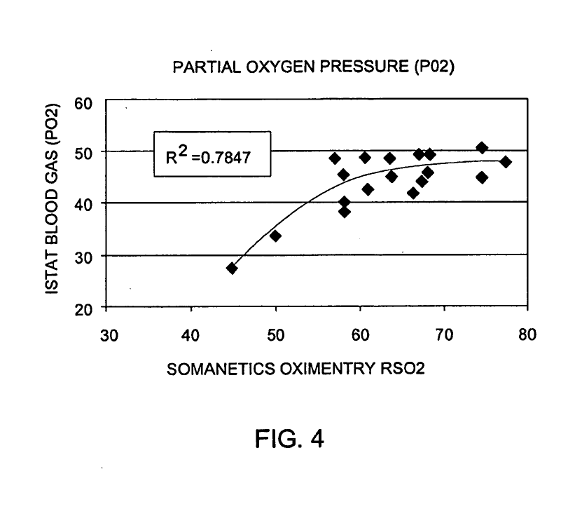

FIG. 3 shows that the percentage of oxygen saturation measured from the NIRS oximetry and the direct blood gas measurement have a strong correlation (R²=0.8714). Also, as shown in FIG. 4, the partial pressure of oxygen (pO₂) measured by NIRS oximetry and the blood gas methods are strongly correlated (R²=0.7847).

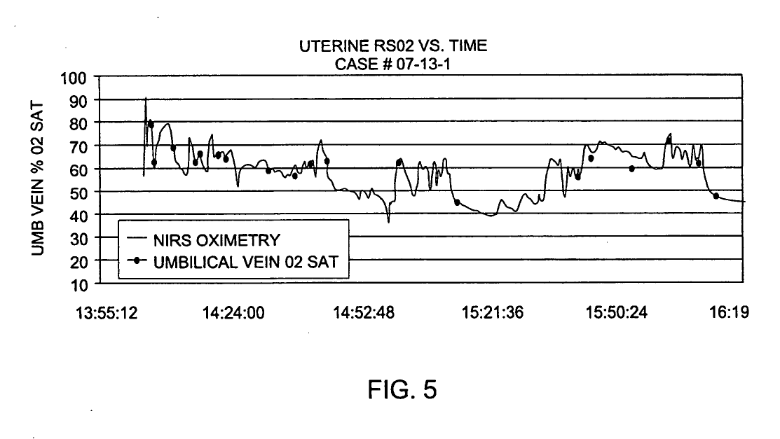

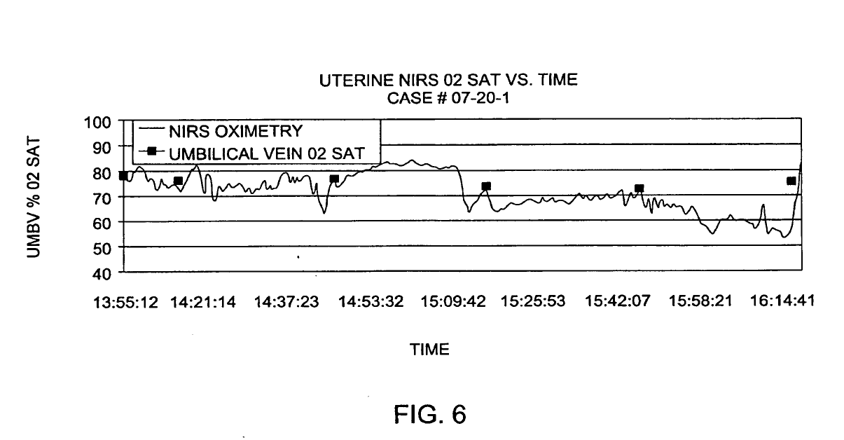

FIGS. 5 and 6 show representative case data for oxygen saturation versus time. As shown, in both cases the blood gas measurements validate the oximetry measurements.

As in the results discussed above, NIRS oximetry data moderately correlates to fetal oxygen saturation and umbilical blood flow. Further, NIRS oximetry does not estimate uterine oxygen saturation; thus, NIRS oximetry measures the fetal, but not the maternal side of the placental circulation.

These findings show that NIRS permits non-invasive assessment of placental oxygen saturation and pO₂. This technology is a simple and useful tool for rapid, real-time monitoring of placental oxygen delivery to the fetus during maternal-fetal interventions and, therefore, can be an effective method of monitoring fetal well-being. The use of the technique can reduce fetal stress and improve fetal outcomes during fetal therapeutics.

In further exemplary embodiments, the sensors are adapted for use in the moist environment of the abdominal cavity. For example, an adhesive appropriate for use in a moist environment is utilized. Also, moisture-resistant insulation may be provided to electrically isolate the sensor connections.

In some embodiments, miniature probes may be utilized. In addition, some embodiments may employ endoscopic insertion or minimally invasive insertion (such as, for example, mini-laparotomy or laparoscopy).

Exemplary embodiments may incorporate a wireless connection to the sensor (such as, for example, Bluetooth® capability). Some exemplary embodiments may utilize nanotechnology and/or capsule technology to provide fetal oxygen saturation measurement capabilities.

In exemplary methods, NIRS may be used to perform oximetry on specific regions of the fetus (for example, the fetus's brain, kidneys, etc.). Additionally, fetal monitoring may be used to complement/supplant current monitoring techniques and monitoring placental oximetry may be used in “high-risk” pregnancies or in “low-risk” pregnant patients that require surgery or other critical care interventions.

While exemplary embodiments of the invention have been set forth above for the purpose of disclosure, modifications of the disclosed embodiments of the invention as well as other embodiments thereof may occur to those skilled in the art. Accordingly, it is to be understood that the inventions contained herein are not limited to the above precise embodiments and that changes may be made without departing from the scope of the invention as defined by the claims. Likewise, it is to be understood that the invention is defined by the claims and it is not necessary to meet any or all of the stated advantages or objects of the invention disclosed herein to fall within the scope of the claims, since inherent and/or unforeseen advantages of the present invention may exist even though they may not have been explicitly discussed herein.

What is claimed is:

1. A method of measuring oxygen concentration within fetal blood comprising:
placing a sensor approximate an outside of a wall of a uterus, the sensor being adapted to be operatively coupled to a near-infrared spectroscopy device; and
measuring a saturation of oxygen in blood of a fetus present within the uterus using the near-infrared spectroscopy device and the sensor.

2. The method of claim 1, wherein the step of placing the sensor includes placing the sensor approximate the outside of the wall of the uterus generally opposing a placenta present within the uterus; and wherein the step of measuring the saturation of oxygen includes measuring a saturation of oxygen in the fetus's blood present within the placenta.

3. The method of claim 1, wherein the sensor is a miniature sensor.

4. The method of claim 3, further comprising, prior to the step of placing the sensor, creating a minimally invasive incision and inserting the sensor through the minimally invasive incision.

5. The method of claim 4, further comprising, prior to the step of placing the sensor, visualizing the uterus using a laparoscope.

6. The method of claim 1, wherein the sensor and the near-infrared spectroscopy device are adapted to be operatively connected via a wireless data link.

7. The method of claim 1, wherein the step of measuring the saturation of oxygen includes continuously measuring the saturation of oxygen during at least a portion of a therapeutic procedure.

8. The method of claim 7, wherein the therapeutic procedure includes placing the fetus on cardiopulmonary bypass.

9. The method of claim 1, wherein the step of placing the sensor includes placing the sensor approximate the outside of the wall of the uterus generally near at least one of a brain and a kidney of the fetus; and wherein the step of measuring the saturation of oxygen includes measuring a saturation of oxygen in the fetus's blood present within at least one of the brain and the kidney.

10. A method of measuring fetal blood oxygen concentration comprising:
inserting a probe into a uterus, the probe including a sensor adapted to be operatively coupled to a near-infrared spectroscopy device; and
measuring a saturation of oxygen in blood of a fetus present within the uterus using the near-infrared spectroscopy device and the sensor.

11. The method of claim 10, wherein the step of inserting the probe includes placing the sensor approximate a placenta present within the uterus; and wherein the step of measuring the saturation of oxygen includes measuring a saturation of oxygen in the fetus's blood present within the placenta.

12. The method of claim 10, wherein the sensor is a miniature sensor.

13. The method of claim 12, further comprising, prior to the step of inserting the probe, creating a minimally invasive incision; wherein the step of inserting the probe includes inserting the probe through the minimally invasive incision.

14. The method of claim 13, further comprising, prior to placing the sensor approximate the placenta, visualizing the uterus using a laparoscope.

15. The method of claim 10, wherein the sensor and the near-infrared spectroscopy device are adapted to be operatively connected via a wireless data link.

16. The method of claim 10, wherein the step of measuring the saturation of oxygen includes continuously measuring the saturation of oxygen during at least a portion of a therapeutic procedure.

17. The method of claim 16, wherein the therapeutic procedure includes placing the fetus on cardiopulmonary bypass.

18. The method of claim 10, wherein the step of inserting the probe includes placing the sensor approximate at least one of a brain and a kidney of the fetus; and wherein the step of measuring the saturation of oxygen includes measuring a saturation of oxygen in the fetus's blood present within at least one of the brain and the kidney.

19. A fetal blood oximetry device comprising:
a near-infrared spectroscopy device; and
a sensor operatively coupled to the near-infrared spectroscopy device;
wherein the sensor is adapted for use at least one of on or within a uterus.

20. The fetal blood oximetry device of claim 19, wherein the sensor includes a tissue-contact surface at least partially covered with an adhesive; and wherein the adhesive is moisture-resistant.

21. The fetal blood oximetry device of claim 19, wherein the sensor includes a connector interposing the sensor and the near-infrared spectroscopy device; and wherein the connector is moisture-resistant.

22. The fetal blood oximetry device of claim 19, further comprising a probe adapted to be inserted into a uterus; wherein the sensor is mounted to the probe.

23. The fetal blood oximetry device of claim 19, wherein the sensor and the near-infrared spectroscopy device are operatively connected via a wireless data link.
